SEQUENCE LISTING (2 sequences)

SEQ ID NO: 1 - protein, 13 aa, synthetic construct
GWYRSPFSRVVHL

SEQ ID NO: 2 - protein, 9 aa, synthetic construct
KYNKANAFL

(19) United States
(12) Patent Application Publication
(10) Pub. No.: US 2026/0083830 A1
(43) Pub. Date: Mar. 26, 2026

(54) BIODEGRADABLE POLYMERIC PARTICLES FOR DELIVERY OF POSITIVELY CHARGED THERAPEUTIC AGENTS

(71) Applicant: REGENTS OF THE UNIVERSITY OF MICHIGAN, Ann Arbor, MI (US)

(72) Inventors: Steven Paul Schwendeman, Superior Township, MI (US); James Moon, Ann Arbor, MI (US); Corrine Din, Ann Arbor, MI (US)

(21) Appl. No.: 19/109,261

(22) PCT Filed: Sep. 6, 2023
(86) PCT No.: PCT/US2023/073594

§ 371 Date: Mar. 6, 2025

Related U.S. Application Data
(60) Provisional application No. 63/374,749, filed on Sep. 6, 2022.

(51) Int. Cl. A61K 39/00; A61K 9/16; A61P 37/06

(52) U.S. Cl. CPC A61K 39/0008; A61K 9/1647; A61K 9/1694; A61P 37/06; A61K 2039/6093; A61K 2039/627

(57) ABSTRACT

The disclosure relates to microparticles and nanoparticles comprising a polymer matrix comprising an uncapped polymer and a net positively charged therapeutic agent at neutral pH. More particularly the disclosure relates to PLGA and/or PLA particles comprising an uncapped polymer for extended, controlled release of positively charged proteins or peptides at neutral pH. Methods of making the particles and administering the particles are also provided.

### INCORPORATION BY REFERENCE OF MATERIAL SUBMITTED ELECTRONICALLY

The Sequence Listing, which is a part of the present disclosure, is submitted concurrently with the specification as an XML file. The name of the XML file containing the Sequence Listing is “58129A_Seqlisting.xml”, which was created on Sep. 5, 2023 and is 2,829 bytes in size. The subject matter of the Sequence Listing is incorporated herein in its entirety by reference.

### FIELD

The present technology relates to biodegradable polymeric particles for extended, controlled release of a positively charged therapeutic agent. More particularly, the disclosure relates to biodegradable polymeric particles for extended, controlled release of a therapeutic agent, in particular, a peptide or protein which has a net positive charge.

### BACKGROUND

Injectable, biodegradable polymeric particles, such as nanoparticles and/or microparticles, provide a means to deliver and control the release of therapeutic agents such as small molecule drugs, proteins, peptides, and antigens (which can be classified as species of proteins or peptides). Once injected, the biodegradable polymeric particles can release the therapeutic agent over the course of hours, days or more extended periods such as weeks or months, thus eliminating the need for daily injections, and thereby improving patient acceptance and compliance as well as outcomes. Controlled release of a therapeutic agent can therefore beneficially reduce the number of doses in an immunization schedule.

Nevertheless, significant obstacles have been encountered. Two significant concerns are the stability of therapeutic agents during encapsulation and high manufacturing costs. Methods for encapsulating therapeutic agents in biodegradable polymers can involve harsh processing conditions, including exposure to organic solvents, high temperatures, homogenization methods such as mixing, sonication, and high-speed agitation, and require aseptic processing. These methods alone or in combination can destabilize therapeutic agents, particularly proteins and peptides, as well as lower loading efficiencies for these therapeutic agents. Micronization of a therapeutic agent prior to encapsulation can further destabilize the therapeutic agent. Finally, conventional methods of encapsulation commonly involve using high concentrations of drug dissolved in water or acetic acid, which can either be destabilizing to the drug or not possible when drug concentrations are above the drug solubility.

According to the National Institute of Health, around 24 million American suffer from at least one of 80 different autoimmune diseases which typically involve autoreactive T cells, such as multiple sclerosis. These autoreactive T cells can cause tissue and organ damage, which can have lasting effects on patients' quality of life. Current approved therapies focus on widespread immunosuppression or general immunomodulatory functions which have shown some success but come with potentially serious side effects and/or modest efficacy (Goodin D. S. et al. Neurology. (2008) 2; 71 (10):766-73; Feinstein, A. et al. Lancet Neurol. (2015) 14(2):194-207; Gasperini C, et al. Drug Des Devel Ther. 6 (2012): 175-86). Recent research efforts related to autoimmune disease treatment have shifted focus to exploring more specific treatment strategies by inducing tolerance using proteins and peptides capable of generating an autoimmune response, also referred to as self-antigen peptides. By delivering a self-antigen peptide, tolerance can be induced by priming the corresponding antigen presenting T cells, and thereby effectively suppressing one or more aspects of the immune response and/or modulate one or more immune functions (Peggs et. al. Clin Exp Immunol. 2009; 157(1):9-19).

Multiple sclerosis (MS) is the most common neurological disease affecting young adults. MS is an autoimmune disease involving over-activation of T cells in the CNS, characterized by loss of motor and sensory functions. There is increasing incidence and prevalence of multiple sclerosis, and its complexities continue to challenge efforts to treat or cure the disease. For example, a common immunomodulating drug used to treat multiple sclerosis is COPAXONE® (glatiramer acetate) which is a copolymer of amino acid residues resembling myelin basic protein (MBP). Although glatiramer acetate showed modest efficacy, relapse-remitting multiple sclerosis patients developed recurrent relapses and some patients developed antibodies against the drug (Brown, Expert opinion on drug delivery, 2 (2005) 29-42). Two other current FDA approved therapies, TYSABRI® (Natalizumab) and GILENYA® (Fingolimod), focus on T cell migration into the CNS. However, these strategies could have severe unintended side effects by introducing general suppression of T cell entry to the CNS. Although side effects with GILENYA® treatment are less severe compared to TYSABRI® treatment, neither therapeutic strategy addresses the underlying cause of multiple sclerosis: the breakdown of T cell tolerance to self-antigens.

In the case of multiple sclerosis, several myelin proteins have been identified as self-antigens capable of triggering autoreactive Th1 and Th17 cells. Fragments of MBP, proteolipid protein, and myelin oligodendrocyte glycoprotein (MOG) have been used to induce experimental autoimmune encephalomyelitis (EAE), a popular animal model of multiple sclerosis, in mice. There are currently several efforts to deliver MOG 35-55 peptides using nanoparticle or microparticle formulations to either focus on T cell anergy or formation of regulatory T cells demonstrating normal immune functions/response (Cho J. et al. Biomaterials 143 (2017) 79-92; Li et al. J Control Release. 331 (2021) 164-175).

Conventional microencapsulation approaches to manufacture PLGA microspheres include solvent evaporation, coacervation, and spray-drying. In each of these methods, the API is combined with PLGA dissolved in an organic solvent before forming microspheres. This combination creates a number of undesirable issues: (a) the peptide-loaded microspheres most often cannot be terminally sterilized, thus requiring expensive aseptic processing with organic solvents and numerous unit operations; (b) yields are often low, which is particularly problematic when the API is expensive (c) products can include one or more residual organic solvents, which pose challenges to storage stability of the final products (van de Weert et. al. Pharmaceutical research, 17 (2000) 1159-1167); (d) there is little opportunity to manipulate the polymer structure once the peptide-PLGA matrix is formed, limiting the ability to engineer release kinetics (Hines, D. J. et al. Crit. Rev. Therapeutic Drug Carr. Syst. 30 (2013) 257-276; Fu, Y. et al. Exp. Opin. Drug Del. 7 (2010) 429-444); and (e) mixing organic solvent/water mixtures in the presence of peptides, particularly with higher-order structure, can be detrimental to drug stability (Schwendeman, S. P. et al. J. Control Release 190 (2014) 240-253). Conventional encapsulation methods also typically require high concentrations of the therapeutic agent (>50-100 mg/mL) in solution in order to achieve adequate loading. This elevated concentration requirement could prohibit encapsulation of therapeutic agents with moderate to low solubility.

The concept of remote loading in aqueous solution was initially shown by encapsulating large molecules including leuprolide and large proteins in porous aliphatic ester end-capped PLGAs, where pores are closed spontaneously by passive healing of the polymer with elevated temperature (Reinhold et. al., Angewandte Chemie Int. Ed., 51 (2012) 10800-10803; U.S. Pat. No. 8,017,155). However, this technique generally requires a “trapping agent” such as aluminum hydroxide adjuvant or dextran sulfate inside the polymer pores to bind the therapeutic agent in order to overcome low encapsulation efficiency (Reinhold et. al., Angewandte Chemie Int. Ed., 51 (2012) 10800-10803). Additionally, incorporation of a water-insoluble base such as MgCO₃ is often required to facilitate continuous drug release (Schwendeman, Recent Advances in the Stabilization of Proteins Encapsulated in Injectable PLGA Delivery Systems. 19 (2002) 26). Achieving both high encapsulation efficiency (e.g., >60%) and very high drug loading (e.g., >5% w/w), particularly when using a relatively low concentration of the therapeutic agent (e.g., less than 30 mg/mL), remains of interest.

Thus, new encapsulation methods that achieve both high encapsulation efficiency (e.g., >60%) and very high drug loading (e.g., >5% w/w) at a relatively low concentration of the therapeutic agent and avoid destabilization of therapeutic agents and lower costs associated with their manufacture are desirable.

### SUMMARY

One aspect of the present invention provides a particle for extended, controlled release of a therapeutic agent, comprising a polymer matrix, wherein the polymer matrix comprises a polymer chosen from poly(lactic-co-glycolic acid) (PLGA), poly(lactic acid) (PLA), or a combination thereof, a therapeutic agent associated with and distributed in and encapsulated by the polymer matrix, wherein the therapeutic agent is net positively charged at neutral pH, wherein at least some of the polymer is uncapped, wherein the uncapped polymer comprises free carboxyl groups at the end of the polymer, wherein the particle have an average particle diameter of about 10 nm to about 10 μm, and wherein the particle has a therapeutic agent content in the range of about 2 weight percent (wt. %) to about 20 wt. %, about 4 wt. % to about 18 wt. %, about 6 wt. % to about 16 wt. %, about 8 wt. % to about 14 wt. %, or about 10 wt. % to about 12 wt. %, based on the entire weight of the particle; and wherein the particle has an initial burst release of the therapeutic agent of about 10% or less after 24 hours in a phosphate-buffered saline buffer.

Another aspect of the present invention provides a particle for extended, controlled release of a therapeutic agent, comprising a polymer matrix, wherein the polymer matrix comprises a polymer chosen from poly(lactic-co-glycolic acid) (PLGA), poly(lactic acid) (PLA), or a combination thereof, a therapeutic agent associated with and distributed in and encapsulated by the polymer matrix, wherein the therapeutic agent comprises one or more positively charged amino acid residues at neutral pH, wherein the positively charged amino acid residues of the therapeutic agent provide a net positive charge at neutral pH such that the net charge of the therapeutic agent is greater than or equal to about +1, wherein at least some of the polymer matrix comprises uncapped polymer, wherein the uncapped polymer comprises free carboxyl groups at the end of the polymer, wherein the particle has an average particle diameter of about 10 nm to about 100 μm, wherein a negatively charged counter ion is coupled to a portion of the positively charged amino acid residues and a conjugate acid of the counter ion has a pKa of about 0.1 to about 4.5, about 0.2 to about 4.0, or about 0.3 to about 3.5.

In another aspect, the invention provides a formulation comprising a plurality of the particles disclosed herein and a pharmaceutically acceptable excipient. Another aspect of the present invention provides a method for treating an autoimmune condition or disease, comprising administering to a subject in need thereof, the particle or formulation disclosed herein.

Another aspect of the present invention provides a method of making a particle for controlled release of a therapeutic agent by providing a particle comprising a polymer matrix, the polymer matrix comprising a polymer chosen from one or more in the group of PLGA and PLA, and incubating the particles with a therapeutic agent in a liquid, thereby encapsulating the therapeutic agent in the polymer matrix, wherein the particle has an average particle diameter in the range of about 10 nm to about 10 μm, wherein at least some of the polymer is uncapped, wherein the uncapped polymer comprises free carboxyl groups such that the particle includes free carboxyl groups, wherein the therapeutic agent has a net positive charge at neutral pH, wherein the particle has a therapeutic agent content of about 2 wt. % to about 20 wt. %, about 4 wt. % to about 18 wt. %, about 6 wt. % to about 16 wt. %, about 8 wt. % to about 14 wt. %, or about 10 wt. % to about 12 wt. %, based on the entire weight of the particle, wherein the encapsulation efficiency of the therapeutic agent is in the range of about 40% to about 100%, about 50% to about 100%, about 60% to about 100%, about 70% to about 100%, about 80% to about 100%, or about 90% to about 100%, and wherein the particle has an initial burst release of therapeutic agent of about 10% or less, after 24 hours in a phosphate-buffered saline buffer.

Another aspect of the present invention provides a method of making a particle for controlled release of a therapeutic agent by providing a particle comprising a polymer matrix, the polymer matrix comprising a polymer chosen from one or more in the group of PLGA and PLA, wherein the particle has an average particle diameter in the range of about 10 nm to about 100 μm, wherein at least some of the polymer is uncapped, wherein the uncapped polymer comprises free carboxyl groups such that the particle includes free carboxyl groups, wherein the therapeutic agent comprises one or more positively charged amino acid residues at neutral pH, such that the net positive charge of the therapeutic agent is greater than or equal to about +1 at neutral pH, wherein a negatively charged counter ion is coupled to a portion of the positively charged amino acid residues, and wherein a conjugate acid of the counter ion has a pKa of about 0.1 to about 4.5, about 0.2 to about 4.0, or about 0.3 to about 3.5, and wherein the encapsulation efficiency of the therapeutic agent is in the range of about 40% to about 100%, about 50% to about 100%, about 60% to about 100%, about 70% to about 100%, about 80% to about 100%, or about 90% to about 100%.

Another aspect of the present invention provides a method of making a particle for controlled release of a therapeutic agent by providing a plurality of particles comprising a polymer matrix, the polymer matrix comprising an uncapped polymer chosen from one or more in the group of PLGA and PLA, incubating the plurality of the particles with a therapeutic agent in an aqueous solvent, thereby encapsulating the therapeutic agent in the polymer matrix and forming a loaded particle, and removing the solvent and drying the loaded particles, wherein the uncapped polymer comprises free carboxyl groups such that the particle includes free carboxyl groups, wherein the therapeutic agent comprises one or more positively charged amino acid residues at neutral pH, wherein the positively charged amino acid residues of the therapeutic agent provide a net positive charge at neutral pH such that the net charge of the therapeutic agent is greater than or equal to about +1, wherein the concentration of the particles during incubation is in the range of about 100 mg/mL to about 300 mg/mL, about 100 mg/mL to about 250 mg/mL, about 100 mg/mL to about 200 mg/mL, about 150 mg/mL to about 300 mg/mL, or about 180 mg/mL to about 240 mg/mL, wherein the concentration of the therapeutic agent during incubation is in the range of about 10 mg/mL to about 20 mg/mL, and wherein the particles are incubated at a temperature in the range of about 35° C. to about 45° C.

Further aspects of the disclosure may become apparent to those skilled in the art from a review of the following detailed description, taken in conjunction with the examples and appended claims. While the invention is susceptible to embodiments in various forms, described herein are specific embodiments of the invention with the understanding that the disclosure is illustrative, and is not intended to limit the invention to specific embodiments described herein.

### BRIEF DESCRIPTION OF THE DRAWINGS

FIG. 1 shows the effect of trehalose on microparticle porosity (A), encapsulation efficiency (B), loading (C), and initial burst release of leuprolide (D).

FIG. 2 shows the effect of loading time on encapsulation efficiency (A) and loading (B) of microparticles prepared with varying amounts of trehalose.

FIG. 3 shows the release profile of leuprolide from loaded microparticles over 45 days.

FIG. 4 shows the slow and continuous release profile of peptides from remote loaded PLGA nanoparticles over 50 days.

FIG. 5 shows the effect of remote loaded PLGA nanoparticles on EAE scores over 77 days.

FIG. 6 shows the effect of remote loaded PLGA nanoparticles on Treg response over 15 days.

### DETAILED DESCRIPTION

The present invention discloses a method for encapsulating therapeutic agents, including but not limited to self-peptides, in biodegradable polymeric particles, such as microparticles and nanoparticles. Advantageously, the nanoparticles and microparticles described herein can efficiently encapsulate therapeutic agents such as self-antigens without significant degradation of the therapeutic agent and continuously deliver the self-antigen in its active form over an extended period of time, e.g., more than three weeks or 21 days, more than four weeks or 28 days, more than five weeks, more than six weeks, more than seven weeks, more than eight weeks, or even longer. These biodegradable polymeric particles can advantageously induce tolerance to various antigens, by injecting these microparticles and/or nanoparticles in the body to slowly and continuously release self-peptide antigens. This technology can be administered to treat, mitigate, and/or ameliorate multiple autoimmune disease therapeutic strategies where enhanced antigen tolerance provides a therapeutic approach to overcoming conditions involving autoreactive T cells, specifically by promoting patient tolerance to self-antigens.

Additionally, by minimizing contact with organic solvents, application of excess heat, and homogenization methods such as mixing, sonication, and high-speed agitation, the present invention provides opportunities to increase stability of the therapeutic agent and scalability as well as clear advantages such as enhanced encapsulation efficiency for reducing costs of goods associated with manufacturing. By using the aqueous loading method described herein, exposure of the therapeutic agent or self-antigen to harsh solvents like methylene chloride or to a micronization step can advantageously be avoided. Loading of the biodegradable polymeric particles disclosed herein also beneficially provides high encapsulation efficiency, high loading of the therapeutic agent, the capability to use a relatively low therapeutic agent concentration in the loading solution, and controlled release functionality to the loaded particles without the need for excipients such as porosigen(s), trapping agent(s) and water-insoluble base(s). This new loading method, which can be accomplished in a simple aseptic aqueous mixing step with terminally sterilized microparticles and nanoparticles, relies on absorption of net positively charged therapeutic agents rather than adsorption as in previous remote loading systems. These microparticles and nanoparticles can feature counter ions which may result in an unexpected improvement in therapeutic agent loading and encapsulation efficiency when combined with uncapped polymers. While not intending to be bound by theory, it is theorized that the counterions are coupled to and/or associated with the positively charged therapeutic agent, as well as with the polymer end groups, as will be explained in further detail below.

The term “about” is used according to its ordinary meaning, for example, to mean approximately or around. In one embodiment, the term “about” means±10% of a stated value or range of values. In another embodiment, the term “about” means±5% of a stated value or range of values. A value or range described in combination with the term “about” expressly includes the specific value and/or range as well (e.g., for a value described as “about 40,” “40” is also expressly contemplated).

### Composition of Polymeric Particle

Natural and synthetic polymers such as poly(lactides), poly(glycolides), poly(lactide-co-glycolides), poly(lactic acid) s, poly(glycolic acid) s, poly(lactic acid-co-glycolic acid) s, polycaprolactone, poly(hydroxymethyl glycolide-co-lactide), polycarbonates, polyesteramides, polyan hydrides, poly(amino acids), polyorthoesters, polycyanoacrylates, poly(p-dioxanone), poly(alkylene oxalate) s, biodegradable polyurethanes, homopolymers, copolymers, and blends of these and other polymers may be used to form polymer matrices as disclosed herein. Among these polymers, poly(lactic-co-glycolic acid) (PLGA)-based polymer matrices and particles possess highly desirable qualities for drug delivery such as biodegradability and biocompatibility. PLGA polymers have been used extensively in microparticles, millicylindrical rods, coatings and various other devices for therapeutic delivery, and their rates of degradation and biocompatibility are well understood. Poly(lactic acid) (PLA)-based polymers also exhibit desirable qualities for drug delivery such as biodegradability and biocompatibility.

The particles of the disclosure feature a polymer matrix, comprised of PLGA, PLA, or a combination thereof. PLGA and PLA based polymers feature carboxyl groups at the end of the polymer. As used herein, the term “uncapped polymer” refers to a polymer with “free” carboxyl groups at the end of the polymer. In many cases, uncapped natural and synthetic polymers can be used to provide the polymer matrix in the particles disclosed herein. Further, the particles disclosed herein generally are substantially free of a “capped polymer” which refers to a polymer in which the carboxyl groups have been substituted or replaced with other functional groups. Typically, in a capped polymer, the carboxyl groups at the end of the PLGA or PLA are replaced with hydrophobic groups, such as ester groups, to facilitate encapsulation of therapeutic agents.

As used herein, the term “substantially free” means that the compositions and/or particles according to the disclosure contain insignificant amounts of the indicated component. For example, the particles according to the disclosure may contain less than 5 weight percent, less 2 wt. %, less than 1 wt. %, or less than 0.10 wt. % of the indicated component, based on the entire weight of the composition or particle.

As set forth above, the biodegradable polymeric particle is comprised of uncapped PLGA or PLA or a combination. In a preferred embodiment, the particle is comprised of uncapped PLGA. The uncapped PLGA polymer may have a lactic acid content in the range of about 25% to about 100%. In preferred embodiments, the uncapped polymer has a lactic acid content of about 75%. In preferred embodiments, the polymer matrix has at least some uncapped polymer such that the particle has carboxyl groups. Generally, at least about 50%, at least about 75%, and/or at least 90% of the polymer in the polymer matrix is uncapped.

In various cases, the uncapped polymer has a weight average molecular weight in the range of about 2 kDa to about 50 kDa, about 3 kDa to about 45 kDa, about 5 kDa to about 40 kDa, about 7.5 kDa to about 35 kDa, or about 10 kDa to 20 kDa. In preferred embodiments, the uncapped polymer has a weight average molecular weight in the range of about 10 kDa to about 20 kDa.

### Therapeutic Agents

Disclosed herein are methods for loading biodegradable polymeric particles with net positively charged therapeutic agents. As used herein, the term “therapeutic agent” refers to a protein, peptide or small molecule drug. In many cases, the therapeutic agent is a protein or a peptide. In preferred embodiments, the therapeutic agent is an antigen. In preferred embodiments, the therapeutic agent is a self-antigen.

As used herein, the term “antigen” refers to a molecule capable of generating an immune response from a subject. As used herein, the term “self-antigen” refers to a protein or peptide which does not act as an antigen in a healthy subject, but is capable of generating an immune response in a subject with an autoimmune condition or disease. In some cases, the self-antigen is MOG 38-50, GWYRSPFSRVVHL (SEQ ID NO:1). In some cases, the self-antigen is NRPA7, KYNKANAFL (SEQ ID NO: 2). As used herein, the term “autoimmune condition or disease” refers to a disease or disorder that interferes with the proper functioning of the immune system, particularly when the immune cells in a subject attack its own healthy cells. It can be chronic pathology triggered by the loss of immunological tolerance to self-antigens, which can cause systemic or organ specific damage. In some instance, autoimmune response is mediated by autoreactive T and B lymphocytes responsible for the production of soluble mediators (e.g., cytokines, nitric oxide, etc.) and autoantibodies. Infections can be a cause of the autoimmune disease or disorder.

In some embodiments, an autoimmune disease or disorder can include but are not limited to Achalasia, Addison's disease, Adult Still's disease, Agammaglobulinemia, Alopecia areata, Amyloidosis, Ankylosing spondylitis, Anti-GBM/Anti-TBM nephritism Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant, cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis (MS), Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy. Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

Self-antigens such as MOG 38-50 and NRPA7 have the potential to provide treatment strategies for autoimmune diseases and conditions that specifically work to reduce T cell recognition of self-antigens through tolerance. T cell tolerance is developed through sustained exposure of self-antigens. Typically, this requires regimented inoculations to maintain exposure. In various cases, the particles of the disclosure can provide sustained release of self-antigens such as MOG 38-50 and NRPA7. In some cases, the therapeutic agent is a vaccine antigen. As used herein, the term “vaccine antigen” refers to a protein or peptide derived from an infectious or communicable disease used to inoculate a subject against said infectious or communicable disease. In some cases, the therapeutic agent is a neoantigen, i.e., an antigen for cancer immunotherapy. In some cases, the therapeutic agent is an antigen for infectious disease.

Generally, the molecular weight of the therapeutic agent can be determined by liquid chromatography with mass spectrometry. In various embodiments, the therapeutic agent has a molecular weight in the range of about 500 Da to about 5 kDa, about 1 kDa to about 4.5 kDa, about 1.5 kDa to about 4 kDa, or about 2 kDa to about 3.5 kDa.

In one aspect, the therapeutic agents of the disclosure can be water-soluble. However, in another aspect, because the particles and methods of the disclosure are capable of high therapeutic agent loading and high encapsulation efficiency, because of the significant structural interactions involved in the particles and methods described herein, the therapeutic agents used in the particles and methods of the disclosure can advantageously demonstrate moderate or low solubility in water, particularly relative to the therapeutic agents used in prior art techniques. Generally, the therapeutic agents used in the particles and methods have a solubility in water of 100 mg/mL or less. In one aspect, the therapeutic agent has a solubility in water of less than 1 mg/mL. In another aspect, the therapeutic agent has a solubility in water in the range of about 1 mg/mL to about 100 mg/mL, about 1 mg/mL to about 90 mg/mL, about 1 mg/ml to about 80 mg/mL, about 1 mg/mL to about 70 mg/mL, about 1 mg/mL to about 60 mg/mL, about 1 mg/mL to about 50 mg/mL, about 1 mg/mL to about 40 mg/mL, about 1 mg/mL to about 30 mg/mL, about 1 mg/mL to about 20 mg/mL, and/or about 1 mg/mL to about 10 mg/mL.

Generally, the therapeutic agents of the disclosure have a net positive charge at neutral pH. In some cases, the therapeutic agent has a net positive charge greater than +1 at neutral pH. In some cases, the therapeutic agent has a net positive charge of about +1 at neutral pH. In preferred embodiments, the net positively charged therapeutic agent features one or more positively charged moieties at neutral pH such that the net charge of the therapeutic agent is greater than or equal to about +1 at neutral pH. For example, the therapeutic agent can have a net charge greater than or equal to about +1.5 at neutral pH, a net charge greater than or equal to about +1.7 at neutral pH, and/or a net charge greater than or equal to about +2 at neutral pH.

In many embodiments, the net positively charged therapeutic agent is a protein or peptide. In some embodiments, the net positively charged therapeutic agent is a self-antigen. In preferred embodiments, the therapeutic agent contains more than one positively charged moieties at neutral pH. In many embodiments, the positively charged moieties are positively charged amino acid residues at neutral pH. For example, the self-antigens MOG 38-50, and NRPA7 both feature amino acid residues that are positively charged at neutral pH (pH=7). In some cases, the positively charged amino acid residue includes one or more of positively charged lysine, arginine, or histidine residues. In some cases, the positively charged moiety is guanidinium, ammonium, or imidazolium.

### Ion-Pairing

In general, such net positively charged therapeutic agents are preferred because they can readily dissociate into solution and the positively charged moieties of these net positively charged therapeutic agents can associate with carboxyl groups from the ends of the uncapped polymer throughout the particle and/or on the surface of the biodegradable polymeric particle as well as with a counter ion as explained in more detail below. The association between the positively charged moieties on the net positively charged therapeutic agent and the carboxyl groups of the ends polymer advantageously facilitate initial absorption of the therapeutic agent into the biodegradable polymeric particle. However, while it was expected that therapeutic agents with a net positive charge greater than or equal to about +2 at neutral pH would decrease the amount of the carboxyl groups on the surface and/or throughout the biodegradable polymeric particle, it was found that including a counterion as disclosed herein advantageously enhanced loading (or therapeutic agent content) as well as encapsulation efficiency. Thus, while positively charged moieties can improve association of the therapeutic agent to the polymer, as the net charge of the therapeutic agent becomes more positive such as +2 or greater, loading and encapsulation efficiency are generally expected to decrease, but loading and encapsulation were surprisingly increased in the particles according to the invention.

In one aspect, the identity of the counter ion may be described using pKa values. The pKa value represents a logarithmic scale of comparing acidity between different compounds. Generally, pKa values are influenced by the relative stability of the conjugate base of the compound. For example, acetic acid has a pKa of about 4.8, while trifluoroacetic acid has a pKa of 0.2. Due to the three fluoro groups on trifluoroacetic acid, the negative charge can be distributed across more of the conjugate base, which makes the parent acid significantly more acidic than the unsubstituted analogue, acetic acid. In preferred embodiments, the counter ion has a pKa in the range of about 0.1 to about 4.5, about 0.2 to about 4.0, or about 0.3 to about 3.5.

While these therapeutic agents are introduced into solution as ionic salts, upon encapsulation some negatively charged counter ions may remain coupled to or associated with the positively charged moieties of the therapeutic agent. The presence of these counter ions is believed to facilitate the encapsulation of net positively charged therapeutic agents into particles comprised of uncapped polymer. Without being bound by theory, it is theorized that the counter-ion-positively charged moiety pair reduces the opportunity for multiple free carboxyl groups from uncapped polymer that are available on/in biodegradable polymeric particle to interact with the positively charged moieties of the therapeutic agent, which allows greater amounts of the net positively charged therapeutic agent to absorb into the polymer phase more readily. This further ensures that the therapeutic agent is associated with, distributed in, and encapsulated by the particle without the need for any pore forming agents. Thus, porosigens are optional. Additionally, this enables the loading method described herein to be used with a variety of net positively charged therapeutic agents while advantageously demonstrating high loading and encapsulation efficiency.

Generally, again, without being bound by theory, it is expected that at least one positively charged moiety of the therapeutic agent can be coupled or associated with a counter ion. In various embodiments, the therapeutic agent comprises at least two positively charged amino acid residues and, while one is believed to be associated with or coupled to the counter ion, at least one of the positively charged amino acid residues is believed not to couple to a counter ion at neutral pH, and is instead believed to be coupled to or associated with a carboxyl group of the polymer particle as previously described. In various embodiments, a portion of the positively charged moieties are coupled with the counter ion. In various embodiments, the negatively charged counter ion is HCOO⁻, or C₁₋₅alkyl-COO⁻. Additionally, the C₁₋₅alkyl-COO⁻ can be optionally substituted with methyl, fluoro, chloro, or bromo. In various embodiments, the negatively charged counter ion is trifluoroacetate or formate. In preferred embodiments, negatively charged counter ion is formate.

### Methods of Making Polymeric Particles

The particles described herein are prepared via a method of either single or double oil-water or water-oil-water emulsion of uncapped polymers. In many cases, the method is a single oil-water emulsion. In many cases, the method is a double water-oil-water emulsion. The particles of the disclosure feature a polymeric matrix comprising an uncapped polymer, typically PLGA, PLA, or a combination thereof. The carboxyl groups of the uncapped polymer are such that the biodegradable particles feature carboxyl groups. Some of these carboxyl groups are able to associate with the positively charged moieties of the therapeutic agent and facilitate absorption of the therapeutic agent into the polymer matrix. These carboxyl groups available to associate with the positively charged moieties of the therapeutic agent are herein referred to as “free carboxyl groups.”

In embodiments, the biodegradable particles of the disclosure have a substantially neutral potential. In preferred embodiments, the zeta potential of the biodegradable particle is less than ±10 mV.

In some cases, particles are prepared using a single oil-water (O/W) emulsion of uncapped polymer. First, the uncapped polymer is dissolved in an organic solvent and an aqueous solution typically comprising polyvinyl alcohol (PVA) is added to create a water-in-oil (w/o) emulsion. The emulsion is then subjected to evaporation, and particles are formed and can be collected and stored frozen (−20° C.) until further use. For example, in accordance with the disclosure, a plurality of particles can be prepared by dissolving an uncapped polymer in an organic solvent, typically a polar aprotic solvent, such as CH₂Cl₂ thereby forming a solution, adding polyvinyl alcohol to the solution, thereby forming a polymer matrix, and removing the organic solvent, thereby forming the plurality of particles, wherein the polymer matrix comprises PLGA and/or PLA, wherein the uncapped polymer comprises free carboxyl groups such that the particle includes free carboxyl groups.

One particle of the disclosure can have a particle size (i.e., diameter), and a plurality of particles can have an average particle size, ranging from about 10 nm to about 10 μm, for example at least about 20, 25, 30, 40, 45, 50, or 55 nm and/or up to about 1, 2, 3, 4, 5, 6, 7, 8, 9 or 10 μm. In some cases, the particles can have a particle diameter, and a plurality of particles can have an average particle diameter, of about 30 nm to about 10 μm, about 100 nm to about 1 μm, or about 500 nm to about 900 nm. For example, particles of the disclosure can have an average particle diameter in the range of about 10 nm to about 10 μm, about 10 nm to about 1 μm, about 100 nm to about 10 μm, about 100 nm to about 1 μm, about 10 nm to about 100 nm, or about 100 nm to about 10 μm.

In some embodiments, the particle is a nanoparticle. As used herein, the term “nanoparticle” refers to a solid or semi-solid particle having a diameter of less than about 2 μm. The nanoparticle of the disclosure can have a particle size (i.e., diameter), and a plurality of nanoparticles can have an average particle size, ranging from about 10 nm to about 2 μm, for example at least about 20, 25, 30, 40, 45, 50, or 55 nm and/or up to about 1 or 2 μm. In embodiments, the nanoparticle can have a particle size, and a plurality of nanoparticles can have an average particle size, of about 30 nm to about 2 μm, about 100 nm to about 1 μm, or about 500 nm to about 900 nm. The particle size can represent a weight-, number-, surface area-, or volume-average size for a particle size distribution of the nanoparticles. Particle sizes can be quantified by SEM images, and quantified using a Master Sizer 2000 laser diffraction particle size analyzer (Master Sizer 2000, Malvern Instruments Ltd. Malvern. UK). Nanoparticles having a spherical shape are referred to as nanospheres.

In some cases, particles are prepared using a double water-oil-water (W/O/W) emulsion of uncapped polymer. First, the uncapped polymer is dissolved in an organic solvent to create a water-in-oil (w/o) emulsion. Optionally, a porosigen may be added to increase the rate of therapeutic agent loading. Next, an aqueous solution typically comprising polyvinyl alcohol (PVA) is added to create the second emulsion. The emulsion is then subject to evaporation, and microparticles are formed and can be collected, sieved, lyophilized, and stored frozen (−20° C.) until further use.

One particle of the disclosure can have a particle size (i.e., diameter), and/or a plurality of particles can have an average particle size, ranging from about 10 μm to about 100 μm, for example at least about 15, 20, 25, 30, 35, 40, or 45 μm and/or up to about 40, 45, 50, 55, 60, 65, 70, 75, 80, 85, or 90 μm. In some cases, the particle can have a particle diameter, and a plurality of particles can have an average particle diameter, of about 10 μm to about 100 μm, about 15 μm to about 90 μm, or about 20 μm to about 80 μm.

In some embodiments, the particle is a microparticle. As used herein, the term “microparticle” means a solid or semi-solid particle having a diameter of less than about 100 μm and greater than about 10 μm. The microparticle of the disclosure can have a particle size (i.e., diameter), and a plurality of microparticles can have an average particle size, ranging from about 10 μm to about 100 μm, for example at least about 15, 20, 25, 30, 35, 40, or 45 μm and/or up to about 40, 45, 50, 55, 60, 65, 70, 75, 80, 85, or 90 μm. In some cases, the particle can have a particle size, and a plurality of particles can have an average particle size, of about 10 μm to about 100 μm, about 15 μm to about 90 μm, or about 20 μm to about 80 μm. The particle size can represent a weight-, number-, surface area-, or volume-average size for a particle size distribution of the microspheres. Particle sizes can be quantified by SEM images, and quantified using a Master Sizer 2000 laser diffraction particle size analyzer (Master Sizer 2000, Malvern Instruments Ltd. Malvern, UK). Microparticles having a spherical shape are referred to as microspheres

### Method of Loading Polymeric Particles

Generally, prior art methods involving loading particles comprising end-capped polymers and use various excipients such as trapping agents, porosigens and/or a metal salt (e.g. water-insoluble base(s)) to enhance loading and release profiles of therapeutic agents. To the contrary, the present method of the disclosure achieves comparable and/or even, surprisingly, significantly enhanced loading (therapeutic agent content), encapsulation efficiencies, and/or release profiles without significant amounts of end-capped polymers and/or without the need for any excipient(s), such as porosigens, trapping agent(s), and water-insoluble base(s), during loading. For example, the particles may be “substantially free of excipients” such that the particle contains less than about 5, 4, 3, 2, 1, 0.5, 0.1, or 0.01 wt % of any excipient, prior to being exposed to an aqueous solution. In various embodiments, the particle is substantially free of an oligosaccharide or a polysaccharide, such as chitosan, a sulfated glycosamino-glycan, a non-sulfated glycosamino-glycan, hyaluronic acid chondroitin sulfate, dextrose sulfate, dextran sulfate, ketran sulfate, heparin, heparin sulfate or combinations thereof, and/or of water-insoluble base(s) such as aluminum hydroxide, aluminum phosphate, potassium phosphate, magnesium carbonate, calcium phosphate or an ionomer gel, and the like. In some embodiments, the particle is substantially free of porosigens.

In various cases, the particles of the disclosure feature a polymer matrix made of at least some uncapped polymer such that the particle have free carboxyl groups which are available to associate with the therapeutic agent.

The therapeutic agent is loaded into the preformed biodegradable polymeric particles by incubating the particles in an aqueous solution of the net positively charged therapeutic agent at about neutral pH. The biodegradable polymeric particles have carboxyl groups on their surface and indeed throughout the particle and can associate with the positively charged moieties of the therapeutic agent. Upon introduction to the aqueous solution, the positively charged moieties of the therapeutic agent can more readily dissociate from their counter ions, however, upon encapsulation, without intending to be bound by theory, it is theorized that some positively charged moieties of the therapeutic agent can associate with and/or couple to counter ion, through an ion-pairing interaction which remains stable in the polymer matrix. These ion-paired positively charged moieties are therefore unavailable for interacting with the free carboxyl groups on the biodegradable polymeric particle and are theorized to surprisingly increase the loading capacity of the therapeutic agent in the particles as more free carboxyl groups remain available for loading.

Association between the free carboxyl groups of the uncapped polymer with the available positively charged moieties of the therapeutic agent is therefore believed to facilitate absorption of the therapeutic agent by the particle. In many cases, the net positively charged therapeutic agent has one or more positively charged moieties coupled with at least one counter ion. This reduces the chance for multiple carboxyl groups on the biodegradable polymeric particle to interact with the positively charged moieties of the therapeutic agent and helps the positively charged therapeutic agent absorb into the polymer. Consequently, this ion-pairing between the positively charged moieties of the therapeutic agent and the negatively charged counter ions surprisingly and advantageously increases loading content and encapsulation efficiency of the therapeutic agent, especially without the need for porosigens

The therapeutic agent content or loading is quantified as:

$$( \frac{Mass of therapeutic agent encapsulated in particles}{Total mass of particles} ) \times 100.$$

The percentage encapsulation efficiency of the therapeutic agent is calculated as:

$$( \frac{Mass of therapeutic agent encapsulated in particles}{Total mass of therapeutic agent in loading solution} ) \times 100.$$

Advantageously, the particles of the disclosure are capable of high therapeutic agent loading. In many cases the loading wt % is in the range of about 2 wt. % to about 20 wt. %, about 4 wt. % to about 18 wt. %, about 6 wt. % to about 16 wt. %, about 8 wt. % to about 14 wt. %, or about 10 wt. % to about 12 wt. %, based on the entire weight of the particle. In preferred embodiments, the particle has a therapeutic content greater than or equal to 8%. Similarly, the methods described herein provide excellent encapsulation efficiency, for example at least about 95%, 90%, 92%, 95%, 98%, or 99% and/or up to about 95%, 98%, 99%, 99.5%, 99.9% or 100% efficiency. In embodiments, the encapsulation efficiency is about 40% to about 100%, about 50% to about 100%, or greater than or equal to 60%, for example, about 60% to about 100%, about 70% to about 100%, about 80% to about 100%, or about 90% to about 100%.

In methods of the disclosure, the concentration of the particles during incubation can be significantly higher than the concentration of the of the therapeutic agent. For example, the concentration of the particles during incubation can be in the range of about 100 mg/mL to about 300 mg/mL, about 100 mg/mL to about 250 mg/mL, about 100 mg/mL to about 200 mg/mL, about 150 mg/mL to about 300 mg/mL, or about 180 mg/mL to about 240 mg/mL.

In methods of the disclosure, the concentration of the therapeutic agent can be lower than the concentration of particles during incubation. For example, the concentration of the therapeutic agent can be in the range of about 10 mg/mL to about 20 mg/mL, or about 10 mg/ml to about 15 mg/mL, or about 15 mg/mL to about 20 mg/mL.

In methods of the disclosure, the particles can be incubated over a range of temperatures, preferably at room temperature or greater. For example, the particles can be incubated at a temperature greater than about 25° C., less than about 60° C., or in a suitable range there between, for example, in a range of about 35° C. to about 45° C.

In accordance with the disclosure, a method of making a particle for controlled release of a therapeutic agent can include providing a plurality of particles comprising a polymer matrix, the polymer matrix comprising an uncapped polymer chosen from one or more in the group of PLGA and PLA, incubating the plurality of the particles with a therapeutic agent in an aqueous solvent, thereby encapsulating the therapeutic agent in the polymer matrix and forming a plurality of loaded particles, and removing the solvent and drying the plurality of loaded particles, wherein the concentration of the particles during incubation is in the range of about 100 mg/mL to about 300 mg/mL, about 100 mg/mL to about 250 mg/mL, about 100 mg/mL to about 200 mg/mL, about 150 mg/mL to about 300 mg/mL, or about 180 mg/mL to about 240 mg/mL, wherein the concentration of the therapeutic agent during incubation is in the range of about 10 mg/ml to about 20 mg/mL, and the particles are incubated at a temperature greater than 25° C.

### Release Profile of Polymeric Particles

Loading the therapeutic agent into biodegradable polymeric particles and associating (e.g., binding or coupling) the therapeutic agent to the polymer and within the particle, as disclosed herein, provides a particle exhibiting high loading and encapsulation efficiency of the therapeutic agent, a desirable release profile, and stability for an extended time period. The biodegradable polymeric particles of the disclosure are capable of continuous release of the therapeutic agent in vitro for >21 days with low initial burst, i.e. the polymeric particles advantageously release less than about 10% of the therapeutic agent in the first 24 hours after administration. Moreover, the biodegradable polymeric particles of the disclosure are able to provide sustained continuous release for at least 3 weeks, for example, for about 4 weeks, about 5 weeks, about 6 weeks, about 7 weeks, about 8 weeks, or even longer.

In some cases, the particles of the disclosure can also advantageously avoid an undesirable initial burst of the drug from the particle. As used herein, “initial burst” or “initial burst release” refers to the amount of therapeutic agent released in the first 24 hours after administration/exposure to physiological conditions, as modeled by the amount of therapeutic agent released from t=0 hours to t=24 hours in a phosphate-buffered saline buffer (pH=7.4). The particles of the disclosure can advantageously have an initial burst release of the therapeutic agent of about 10% or less in the first 24 hours in a phosphate-buffered saline buffer. For example. particles of the disclosure can have an initial burst release of the therapeutic agent in the range of about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, in the 24 hours in a phosphate-buffered saline buffer.

Additionally, the biodegradable polymeric particles of the disclosure demonstrate a substantially zero-order release profile. A substantially zero-order release profile refers to a release profile (i.e., a release rate) that is substantially constant over a period of time. As used herein, the term “substantially zero-order release profile” means that the rate of release of the therapeutic agent from the particle does not vary by more than about 100%, e.g., no more than about 35% over the lifetime of the particle. In one aspect, the term substantially zero-order release profile refers to a release profile in which about 10 wt. % to about 20 wt. % of the therapeutic agent (relative to the original amount of therapeutic agent in the particle) is released per week for at least 3 weeks, for at least 4 weeks, for at least five weeks, for at least six weeks, for at least seven weeks, and/or at least eight weeks.

### Methods of Administration

The polymeric of the disclosure can be used in injectable compositions. For example, the disclosure provides injectable formulations for parenteral administration including the biodegradable polymeric particles of the disclosure. The formulations can further include a pharmaceutically acceptable excipient. Suitable routes for parenteral administration include intravenous, subcutaneous, intradermal, intramuscular, intraarticular, and intrathecal. In one aspect, subcutaneous administration is preferred. Advantageously, the biodegradable polymeric particles of the disclosure can provide a route for subcutaneously administering therapeutic agents such as self-antigens with controlled release thereof such that delivery can be adjusted to stop at, or after, a predetermined period of time when a desired effect has been achieved. Additionally, the biodegradable polymeric particles of the disclosure can provide a new route for inoculating against infectious and communicable diseases with vaccine antigens.

The pharmaceutically excipient can include sterile water, saline, or a buffered solution. Additional excipients can include, but are not limited to carboxymethylcellulose sodium, D-mannitol, polysorbate, and combinations thereof, which can be added to help resuspension of the polymeric particles. The polymeric particles can advantageously be injected to a subject via incorporation into a microneedle.

### EXAMPLES

The following examples demonstrate the preparation and loading of uncapped PLGA microparticles and nanoparticles with positively charged therapeutic agents (Examples I and III), as well as evaluate the effect of microparticle porosity on loading and encapsulation efficiency of charged therapeutic agents (Example II), size and surface morphology variations of the nanoparticles (Example IV), in-vitro release profiles of positively charged therapeutic agents from the microparticles and nanoparticles (Example V), and evaluate the effects of ion pairing on remote of therapeutic agents into microparticles and nanoparticles (Example VI) as well as in vivo release studies of self-antigens with nanoparticles (Examples VII, VIII, and IX).

### Materials

MOG 38-50 (GWYRSPFSRVVHL) and NRPA7 (KYNKANAFL) peptides were purchased from Genemed Synthesis (San Antonio, TX) as either trifluoracetate (TFA) or acetate salts as noted below. Leuprolide acetate was purchased from SHNJH Pharmaceuticals (Shanghai, China). Uncapped PLGA (75/25 D,L lactic/glycolic ratio) with molecular weight 13 kDa was purchased from Wako Chemicals (Osaka, Japan). Poly vinyl alcohol (PVA-88% hydrolyzed) was purchased from Sigma Aldrich (St. Louis, MO). Uncapped PLGA (50/50 D,L lactic/glycolic ratio) (Resomer® RG 503H) was purchased from Evonik (Essen, Germany). Hydroxyethyl-piperazineethanesulfonic acid (HEPES) was purchased from Thermo Fisher Scientific (Waltham, MA). All other materials were of analytical grade and purchased from commercial suppliers.

The surface morphology of PLGA microspheres (loaded and unloaded) were analyzed by gold coating (Polaron Sputter Coater) for 90 s followed by imaging with a scanning electron microscope (AMRAY 1910 Field Emission Scanning Electron Microscope), using a gun voltage of 5 kV.

Nanoparticle size was determined by dynamic light scattering (DLS) using a Malvern Zetasizer. The loading percentage and encapsulation efficiency for the nanoparticle formulation was determined by a multiple extraction protocol and UPLC. Additionally, 5 mg of each formulation was aliquoted into 2 mL round bottom Eppendorf tubes with 0.5 mL of PBS and 0.02% Tween 80 at pH 7.4 and placed in an incubator at 37° C. At predetermined time points, samples were centrifuged at 8000 rpm for 5 min and 0.4 mL was collected for analysis by UPLC for determination of release kinetics.

### Example I—Preparation and Loading of Microparticles

PLGA microparticles were prepared by double water-oil-water (W/O/W) emulsion. The first emulsion was created by dissolving PLGA (50/50) (800 mg) in methylene chloride (1 mL). Once dissolved, 200 μl of a 500 mg/mL trehalose in diH₂O solution was added to the polymer solution and then homogenized (VirTis Tempest I.Q.) for 1 minute at 10,000 rpm. Next, 4 mL of a 5% poly vinyl alcohol (PVA) solution was added to the emulsion. The second emulsion was created by vortexing (Scientific Industries Vortex Genie 2) for 1 minute at maximum speed. The w/o/w was then added to a 100 mL stirring bath of 0.5% PVA and stirred for 3 hours for solvent evaporation. After hardening, microparticles were washed extensively with diH₂O and sieved for size in the range 20-63 μm. Microparticles were lyophilized (Labconco FreeZone 2.5) and stored frozen (−20° C.) until further use.

Leuprolide loading solution of 3.6 mg/ml leuprolide acetate was made by dissolving the peptide in a 0.1 M HEPES (pH 7.4) solution. A final loading solution volume of 2 mL was attained by adding diH₂O at a volume necessary to achieve 2 mL after titration.

Pre-formed blank PLGA microparticles were loaded with a solution of leuprolide in the aforementioned HEPES buffer solution. Remote loading was done by incubating 14 mg of microparticles with leuprolide loading solution (1 mL) at 37° C. with mixing for 24 h. Dispersed microparticles were incubated at 37° C. and mixed for 24 h. After incubation, microparticles were centrifuged (Eppendorf 5424R) for 10 minutes at 5,000 rpm and the supernatant was collected. Microparticles were next washed three times with 1 mL diH₂O and the supernatant was saved; centrifuging at 5,000 rpm for 10 minutes between each wash. Loaded and washed microparticles were then lyophilized to remove excess water and stored at −20° C. until future use.

### Example II—Effects of Particle Porosity on Loading & Encapsulation Efficiency of Microparticles

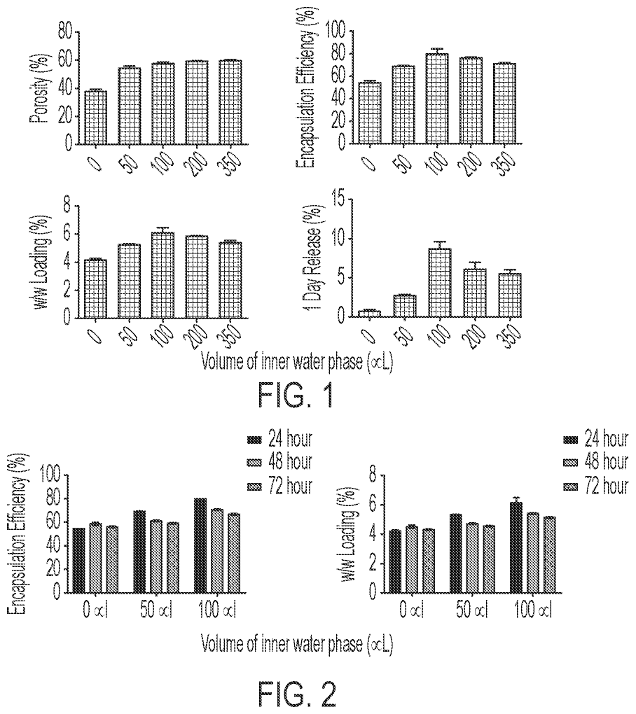

Porosity is typically considered a key parameter of controlled release microparticles as it can affect both therapeutic agent loading and drug release rate. Uncapped PLGA (50/50) microparticles with increasing amounts of trehalose showed an increase in porosity from 38%-60% (FIG. 1A), with the greatest increase in porosity occurring between 0 μl to 50 μl added trehalose solution. Microparticles of increasing trehalose content were loaded with leuprolide at a polymer concentration of 240 mg/ml; the encapsulation efficiency (FIG. 1B) and loading (FIG. 1C) increased with increasing porosigen from 0 μl-100 μl.

After 100 μl trehalose, the encapsulation and loading was similar for the microparticles containing 100 μl to 350 μl trehalose at ˜70% encapsulation and ˜5.8% loading. These results further support that the peptide is being absorbed into the polymer phase as increasing the porosity did not affect the encapsulation particularly for the 100 μl to 350 μl trehalose microparticles. Increasing the porosity did, however, increase the percent leuprolide released in 1 day relative to the 0 μl-100 μl added trehalose microparticles (FIG. 1D).

To test the effect of porosity, microparticles prepared with 0, 50, and 100 μL inner-water phase volumes were loaded over a longer time period, for 48 and 72 hours. However, leuprolide loading does not improve with longer loading period but rather starts to decrease in the case of 50 μL and 100 μL trehalose (FIG. 2).

This contrasts with remote loading via passive self-healing into percolating pores as described in US 2012/0288537. In the present disclosure, loading is indifferent to polymer porosity because the mass of sorbent is kept constant, whereas previous encapsulation systems depend strongly on the porosity as more pores provide additional space for encapsulation on a constant preformed microparticle weight basis. This surprising feature of uncapped PLGA microparticles is due to the fact that leuprolide is not adsorbing to the particle as in previous systems, rather it is theorized that leuprolide is being absorbed by the polymeric particle.

### Example III—Preparation and Loading of Nanoparticles

Unlike the PLGA microparticles exemplified in Example I, PLGA nanoparticles were prepared by a single water-oil (W/O) emulsion method. Uncapped PLGA (72/25) at 3.5% w/v was dissolved in dichloromethane and 5% w/v PVA was added prior to single emulsification using a homogenizer. The resulting emulsion was then transferred into a 0.5% PVA bath and mixed for 3 h to allow for solvent evaporation. After mixing, nanoparticles were centrifuged for collection and washed 3 times with water before freeze drying for at least 24 h.

The resulting blank PLGA nanoparticles were then incubated with an aqueous therapeutic agent loading solution at 37° C. for 24 h to form nanoparticles loaded with therapeutic agent. These loaded nanoparticles were centrifuged for collection, washed 3 times with water, and freeze dried. The loading percentage and encapsulation efficiency for the loaded nanoparticles was determined by a multiple extraction protocol and UPLC. At predetermined time points, samples were centrifuged at 8000 rpm for 5 min and 0.4 mL was collected for analysis by UPLC for determination of release kinetics.

Remote loading of net positively charged self-antigens, such as MOG 38-50 and NRPA7, using uncapped PLGA allows for simple therapeutic agent encapsulation at competitive loading and encapsulation efficiencies without the need for additional excipients like dextran sulfate or aluminum adjuvants used in remote encapsulation during loading. Blank PLGA (72/25) nanoparticles were loaded with MOG 38-50 at 9.5±0.3%, giving rise to greater than 90% encapsulation efficiency. NRPA7 was loaded in blank PLGA nanoparticles at 10.4%+0.6 giving rise to roughly 100% encapsulation efficiency.

Typical encapsulation via solvent evaporation requires significant levels of therapeutic agent if high loading is desired. However, the remote loading technique described herein has many potential advantages for encapsulation of self-antigens on the small scale, as one could load μg quantities of therapeutic agents as only the desired particle concentration would be necessary for high loading and efficient encapsulation.

### Example IV—Size and Morphological Evaluation of Nanoparticles

Following the procedure of Example III, the volume average size of the nanoparticles prepared with 3.5% w/v PLGA (72/25) concentration was measured at 701.5±30 nm. The dispersibility of the nanoparticles was confirmed by measuring a similar size and polydispersity index before and after lyophilization. The self-antigen loaded nanoparticles also exhibited similar size and dispersibility as compared to the blank nanoparticles with a volume average size of 744.8±199 nm.

The nanoparticles feature smaller sizes than previously known using remote loading methods. This is due to the single emulsion process described in Example III. Additionally, the nanoparticles can be synthesized in one step, lowering the cost of manufacture compared to traditional double-emulsion techniques.

### Example V—In Vitro Release Studies of Microparticles and Nanoparticles

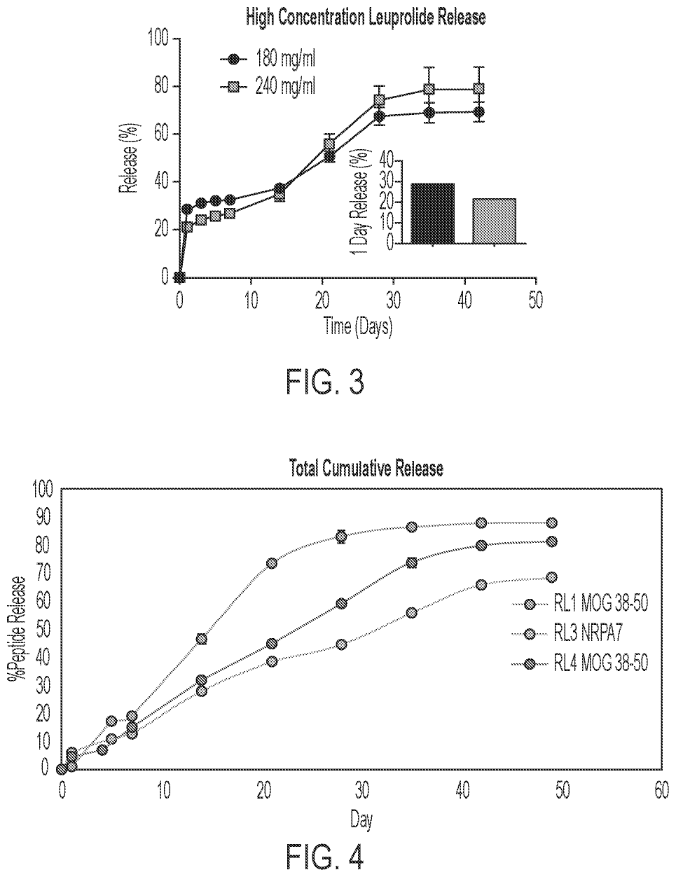

Microparticles were loaded with leuprolide using two different concentrations of polymer, 180 mg/mL and 240 mg/mL (FIG. 3). Microspheres loaded from 180 mg/mL exhibited a higher initial burst release (˜30%) relative to those loaded at 240 mg/ml (˜20%). However, while both concentrations produced microparticles that released >60% of the leuprolide by 45 days, particles produced from the 240 mg/mL concentrated solution exhibited about 80% release by 45 days.

Nanoparticles incubated with MOG 38-50 and NRPA7 self-antigens (TFA salts) were prepared and tested for in vitro release. 5 mg of each formulation was transferred into 2 mL round bottom Eppendorf tubes with 0.5 mL of PBS and 0.02% Tween 80 at pH 7.4 and placed in an incubator at 37° C. At predetermined time points, samples were centrifuged at 8000 rpm for 5 min and 0.4 mL was collected for analysis by UPLC for determination of release kinetics. These data are presented in FIG. 4.

Both MOG 38-50 and NRPA7 loaded PLGA (72/25) nanoparticles exhibited slow and continuous release over the course of 56 days. Two batches of MOG 38-50 formulations released 88% and 82% of the encapsulated self-antigen over the release study, and the NRPA7 formulation released 72% of the encapsulated self-antigen. Both MOG 38-50 and NRPA7 formulations exhibited a desirable minimal burst release with less than 10% of the self-antigen released after one day, a common issue with therapeutic agent loaded nanoparticles.

The release profile of the uncapped PLGA loaded microparticles and nanoparticles exhibited near zero-order kinetics with similar low initial burst release and extended release profiles to prior self-encapsulation systems. Thus, uncapped PLGA loaded particles provide a particle for drug delivery with comparable loading and release, without the need for porosigens and more efficient synthesis methods.

### Example VI—Effects of Ion Pairing on Remote Loading

Previous studies on uncapped PLGA and net positively charged therapeutic agents demonstrated that the negatively charged PLGA and positively charged therapeutic agent can interact during loading (Sophocleous et. al. J. Controlled Release, 172, 662-670 (2013)). However, leuprolide which has a ˜+1 charge at neutral pH was absorbed more readily than octreotide, which has a ˜+1.7 charge at neutral pH. This additional charge is expected to cause the therapeutic agent to associate with more than one polymer chain in the PLGA particle and thus decrease the amount of binding sites available for the peptide and consequently reduce encapsulation efficiency and loading.

Analogously, MOG 38-50 has two permanent positive charges in its sequence owing to 2 Arginine residues. NRPA7 has ˜2 positively charged free amino groups owing to the 2 Lysine residues in its sequence. Because of the >+1 charge, the expectation is for the MOG 38-50 self-antigen to have far lower than 100% encapsulation efficiency, as observed with microparticles using peptides with either 2 Arginine residues or 2 free amino groups as the only ionizable groups in the peptide at neutral pH.

However, as demonstrated in Example III, both MOG 38-50 and NRPA7 were loaded into uncapped PLGA (72/25) nanoparticles with surprisingly high loading and encapsulation efficiency. The unexpected results between these two net positively charged self-antigens was further investigated by comparing the identity of the counter-ion to the standard conventional acetate counterion.

In order to determine the effect of counter ions on remote loading and encapsulation efficiency, nanoparticles were loaded with ionic salts of MOG 38-50, a net positively charged self-antigen, featuring trifluoroacetate (TFA) or acetate as the corresponding counter ion and loading was conducted as described in Example III. These data are presented in Table 1, while a select few of these samples are shown in FIG. 4. Comparison of loading and encapsulation efficiencies of TFA and acetate salts of the MOG 38-50 self-antigen surprisingly demonstrated that the identity of the counter ion plays a significant and unexpected role in the remote loading and encapsulation efficiency of the given self-antigen.

TABLE 1
Remote loaded nanoparticle loading and encapsulation efficiencies
for net positively charged self-antigens, MOG 38-50 and
NRPA7, and trifluoroacetate (TFA) or acetate as the counter
ion. The nanoparticles were loaded with MOG 38-50, except
for RL3 which was loaded with NRPA7.

| | | | Encapsulation |
|---|---|---|---|
| Sample | Counter ion | Loading % ± SEM | Efficiency |
| RL1 | TFA | 9.5 ± 0.3% | 95% |
| RL3 | TFA | 10.4 ± 0.6% | 101% |
| RL4 | TFA | 8.7 ± 1.2% | 87% |
| RL7 | Acetate | 4.9 ± 0.1% | 49% |
| RL8A | Acetate | 5.0 ± 0.3% | 50% |
| RL8B | Acetate | 4.9 ± 0.1% | 49% |
| RL9 | Acetate | 5.8 ± 0.5% | 58% |
| RL11 | Acetate | 7.5 ± 0.9% | 75% |
| RL12-1 | Acetate | 7.1 ± 0.6% | 71% |
| RL13 | Acetate | 7.8 ± 0.2% | 78% |

With TFA as the counter ion, encapsulation efficiency was ˜87-100%, while with acetate as the counterion, encapsulation efficiency was lower and between 49-78% (Table 1). Without intending to be bound by theory, it is theorized that the improvement in encapsulation efficiency for TFA over acetate is attributable to ion-pairing between the negatively charged TFA and the positively charged amino acid residues of the net positively charged self-antigen.

This unexpected combination of uncapped PLGA particles loaded with ion-paired salts of net positively charged therapeutic agent (e.g., self-antigens) provides a new strategy to load and encapsulate net positively charged self-antigens with high loading and encapsulation efficiency. Thus, this aqueous loading method can be adopted for a variety of net positively charged self-antigens previously unavailable, by selecting the appropriate counter ion of the therapeutic agent salt.

### Example VII—In-Vivo Study of Remote Loaded Nanoparticles

EAE was induced in a murine model and successfully treated using the particles according to the disclosure. Specifically, C57BL/6J mice were subcutaneously given 200 μL of an emulsion containing 0.5 mg/mL MOG 35-55 peptide and 1.25 mg/ml of myobacterium tuberculosis in complete Freund's adjuvant in order to induce EAE, a model of MS. Each mouse was also given 175 ng of pertussis toxin both with the emulsion and again two days after giving the emulsion. There were 3 treatment groups and one group that only received phosphate-buffered saline (PBS).

For the treatment groups, 100 μg of self-antigen in 1 mL PBS was aliquoted from the MOG 38-50 loaded PLGA nanoparticles batch based on loading % determined by UPLC. The first treatment group received 100 μL of the nanoparticles in PBS two weeks prior to the induction of EAE. The second treatment group received 100 μL of the nanoparticles in PBS one week prior to the induction of EAE. The third treatment group received 100 μL of the nanoparticles in PBS when each individual mouse showed a clinical score of 1 or 2. The mice were then monitored daily using a 0 to 5 clinical score scale, (0=no signs of paralysis/disease, 1=completely limp tail, 2=hind leg weakness, 3=complete paralysis of hind legs, 4=complete paralysis of hind legs and partial paralysis of front legs, 5=moribund). If mice recorded clinical scores of at least 4 for more than 2 days in a row, the mice were euthanized.

For the group that did not receive treatment, 4 out of the 5 mice exhibited paralysis in their hind legs with clinical scores between 2 and 3 within 12 days. These mice continued to show disease progression and were euthanized within 19 days post induction of EAE. For the group that received treatment once clinical scores of 1 or 2 were observed, 4 out of 5 of the mice were treated by Day 11 or Day 12. Once treated, none of these mice recorded clinical scores greater than 2 and these mice maintained steady strength in tails and hind legs. For the group that received treatment 2 weeks prior to EAE induction, 5 out of 6 of the mice have scored no greater than 1.5, and the last mouse scored no greater than a 2.5 with signs of recovery. Finally, for the group that received treatment 1 week prior to EAE induction, 4 out of 5 of the mice have scored no greater than 1.5. The last mouse in this group scored as high as 4; however, the mouse showed signs of recovery and did not score at a 4 for more than one day.

Administration of uncapped PLGA nanoparticles formed and loaded as exemplified above, results in markedly lower clinical score compared to the control group (PBS only). Additionally, administration of self-antigen MOG 38-50 loaded PLGA nanoparticles either 1 or 2 weeks prior exhibited symptoms associated with less severe immune response. Moreover, administration of MOG 38-50 loaded PLGA nanoparticles once symptoms had been present resulted in improvement for 80% of the study group. These results highlight the effective treatment strategies provided by the current disclosure regarding controlled and sustained self-antigen release.

### Example VIII—In-Vivo Study of Intravenous and Subcutaneous Administration of Remote Loaded Nanoparticles

The effect of intravenous versus subcutaneous routes of administration of remote loaded nanoparticles on EAE was evaluated. EAE was induced in a murine model as described in Example VII, except 200 uL of an emulsion containing 1.0 mg/mL MOG 35-55 peptide and 2.50 mg/mL of Mycobacterium tuberculosis in complete Freund's adjuvant was used. The concentration of MOG 35-55 peptide and Mycobacterium tuberculosis was increased to assess the effect of administration route of remote loaded nanoparticles on EAE. There were 3 treatment groups and one group that only received phosphate-buffered saline (PBS).

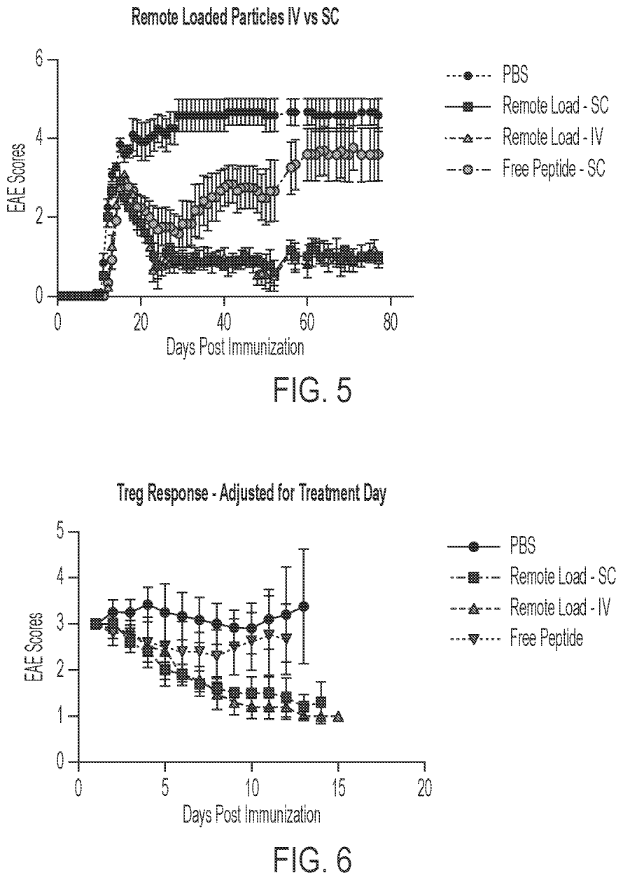

For the treatment groups, 100 μg of self-antigen in 1 mL PBS was aliquoted from the MOG 38-50 loaded PLGA nanoparticles batch based on loading % determined by UPLC. The first treatment group received remote loaded nanoparticles injected subcutaneously by the tail base. The second treatment group received remote loaded nanoparticles injected intravenously in the tail. The third treatment group received 100 ug of free MOG 38-50 peptide injected subcutaneously by the tail base. EAE was assessed daily using the 0 to 5 clinical score scale described in Example VII. The daily EAE clinical scores for the 3 treatment groups and the control group are presented in FIG. 5.

For the group that did not receive treatment, disease progression was demonstrated. By day 15, these mice exhibited clinical scores that steadily increased to 4 and did not improve. For the third treatment group, the EAE clinical score decreased initially, but 30 days after treatment with the free peptide, the EAE clinical scores steady increased. After 60 days, the EAE clinical scores for the third treatment group were observed to be 3.5. For the first and second treatment groups, EAE clinical scores increased to 3.0 until day 15. After day 15, the EAE clinical scores decreased and by day 20, the EAE clinical scores were observed to be 1.5 or less.

Administration of PLGA nanoparticles formed and loaded as exemplified above, results in markedly lower clinical score compared to the control group (PBS only). Additionally, administration of PLGA nanoparticles remote loaded with the self-antigen MOG 38-50 result in a reduction of EAE scores under 1.5 through 60 days of observation. Moreover, regardless of the route of administration, both intravenous and subcutaneous administration of self-antigen MOG 38-50 loaded PLGA nanoparticles resulted in lower EAE scores through 60 days of observation. These results highlight the variety of administration routes provided by the current disclosure regarding controlled and sustained self-antigen release.

### Example IX—In-Vivo Study of Effects of Remote Loaded Nanoparticles on Treg Response

EAE was induced in a murine model as described in Example VIII. There were 3 treatment groups and one group that only received phosphate-buffered saline (PBS). For the treatment groups, 10 ug of self-antigen in 1 mL PBS was aliquoted from the MOG 38-50 loaded PLGA nanoparticles batch based on loading % determined by UPLC. The first treatment group received remote loaded nanoparticles injected subcutaneously by the tail base. The second treatment group received remote loaded nanoparticles injected intravenously in the tail. The third treatment group received 10 ug of free MOG 38-50 peptide injected subcutaneously by the tail base. EAE was assessed daily using the 0 to 5 clinical score scale described in Example VII. The daily EAE clinical scores for the 3 treatment groups and the control group are presented in FIG. 6.

For the group that did not receive treatment, no reduction in EAE scores was observed through 14 days after treatment. During the observation period, the EAE scores of the mice did not decrease below 3.0. For the third treatment group, the EAE scores decreased initially to ˜2.5 at day 8. After day 8, the EAE scores increased to ˜3.0, and did not decrease. For the first and second treatment groups, EAE scores decreased to an average score of ˜1.0 after 15 days. The first and second treatment groups were the only groups that demonstrated decreases in EAE scores over the entire observation range.

Administration of PLGA nanoparticles formed and loaded as exemplified above, results in markedly lower clinical score compared to the control group (PBS only). Additionally, administration of PLGA nanoparticles remote loaded with the self-antigen MOG 38-50 result in a reduction of EAE scores under 1.5 through 15 days. Moreover, regardless of the route of administration, both intravenous and subcutaneous administration of self-antigen MOG 38-50 loaded PLGA nanoparticles resulted in lower EAE scores through 14 days of observation. These results highlight the variety of administration routes provided by the current disclosure regarding controlled and sustained self-antigen release.

1. A particle for extended, controlled release of a therapeutic agent, comprising:
a polymer matrix, wherein the polymer matrix comprises a polymer chosen from poly(lactic-co-glycolic acid) (PLGA), poly(lactic acid) (PLA), or a combination thereof;
a therapeutic agent associated with and distributed in and encapsulated by the polymer matrix, wherein the therapeutic agent is net positively charged at neutral pH;
wherein at least some of the polymer is uncapped;
wherein the uncapped polymer comprises free carboxyl groups at the end of the polymer;
wherein the particle has an average particle diameter in the range of about 10 nm to about 10 μm;
wherein the particle has a therapeutic agent content in the range of about 2% weight percent (wt. %) to about 20% wt. %, based on the entire weight of the particle; and
wherein the particle has an initial burst release of the therapeutic agent of about 10% or less after 24 hours in a phosphate-buffered saline buffer.

2. The particle of claim 1, wherein the uncapped polymer comprises PLGA.

3. The particle of claim 1, wherein the uncapped polymer has a lactic acid content in the range of 25% to 100% and a molecular weight in the range of about 2 kDa to about 50 kDa.

4. The particle of claim 1, wherein the uncapped polymer is PLGA and has a lactic acid content of about 75% and a molecular weight in the range of about 10 kDa to about 45 kDa.

5. The particle of claim 1, wherein the therapeutic agent is a protein, peptide, or small molecule drug.

6. The particle of claim 1, wherein the therapeutic agent has a molecular weight in the range of about 500 Da to about 5 kDa.

7. (canceled)

8. (canceled)

9. The particle of claim 7, further comprising a counter ion, wherein a conjugate acid of the counter ion having a pKa in the range of about 0.1 to about 4.5.

10. (canceled)

11. The particle of claim 8, wherein the counter ion comprises HCOO⁻, or C₁₋₅alkyl-COO⁻, the C₁₋₅alkyl-COO⁻ optionally substituted with a substituent chosen from one or more in the group of methyl, fluoro, chloro, or bromo.

12. The particle of claim 1, wherein the counter ion is formate or trifluoroacetate.

13. (canceled)

14. (canceled)

15. The particle of claim 1, wherein:
the uncapped polymer is PLGA;
the therapeutic agent has a net charge greater than or equal to about +1 at neutral pH, wherein the therapeutic agent comprises at least one positively charged amino acid residue coupled with a negatively charged counter ion, wherein a conjugate acid of the counter ion has a pKa in the range of about 0.1 to about 4.5, about 0.2 to about 4.0, or about 0.3 to about 3.5;
the particle has an average particle diameter in the range of about 500 nm to about 900 nm; and,
the particle has a therapeutic agent content greater than about 8 wt % based on the entire weight of the particle.

16. The particle of claim 1, wherein:
the uncapped polymer is PLGA;
the therapeutic agent has a net charge greater than or equal to about +1 at neutral pH, wherein the therapeutic agent comprises at least one positively charged amino acid residue coupled with formate or trifluoroacetate as a counter ion;
the particle has an average particle diameter in the range of about 500 nm to about 900 nm; and,
the particle has a therapeutic agent content greater than about 8 wt % based on the entire weight of the particle.

17. (canceled)

18. (canceled)

19. (canceled)

20. (canceled)

21. (canceled)

22. (canceled)

23. (canceled)

24. (canceled)

25. (canceled)

26. (canceled)

27. The particle of claim 1, having a substantially zero-order release profile.

28. The particle of claim 1, wherein the particle has a substantially neutral zeta potential.

29. The particle of claim 1, wherein therapeutic agent has moderate to low solubility in water.

30. (canceled)

31. The particle of claim 1, wherein the therapeutic agent has a net charge greater than or equal to about +1.5 at neutral pH.

32. A formulation comprising a plurality of the particles of of claim 1 and a pharmaceutically acceptable excipient.

33. A method of treating an autoimmune condition or disease, comprising administering to a subject in need thereof, the particle or formulation of claim 1.

34. (canceled)

35. (canceled)

36. (canceled)

37. A method of making a particle for controlled release of a therapeutic agent, comprising:
providing a particle comprising a polymer matrix, the polymer matrix comprising a polymer chosen from one or more in the group of PLGA and PLA; and,
incubating the particles with a therapeutic agent in a liquid, thereby encapsulating the therapeutic agent in the polymer matrix;
wherein the particle has an average particle diameter in the range of about 10 nm to about 10 μm;
wherein at least some of the polymer is uncapped;
wherein the uncapped polymer comprises free carboxyl groups such that the particle includes free carboxyl groups;
wherein the therapeutic agent has a net positive charge at a neutral pH;
wherein the particle has a therapeutic agent content in the range of about 2% weight percent (wt. %) to about 20% wt. %, about 6 wt. % to based on the entire weight of the particle; and,
wherein the encapsulation efficiency of the therapeutic agent is in the range of about 40% to about 100%; and
wherein the particle has an initial burst release of therapeutic agent of about 10% or less, after 24 hours in a phosphate-buffered saline buffer.

38. (canceled)

39. (canceled)

40. (canceled)

41. (canceled)

42. A method of making a particle for controlled release of a therapeutic agent, comprising:
providing a plurality of particles comprising a polymer matrix, the polymer matrix comprising an uncapped polymer chosen from one or more in the group of PLGA and PLA;
incubating the plurality of the particles with a therapeutic agent in an aqueous solvent, thereby encapsulating the therapeutic agent in the polymer matrix and forming a plurality of loaded particles; and
removing the solvent and drying the plurality of loaded particles;
wherein:
the uncapped polymer comprises free carboxyl groups such that the particle includes free carboxyl groups;
wherein the therapeutic agent comprises one or more positively charged amino acid residues at neutral pH;
wherein the positively charged amino acid residues of the therapeutic agent provide a net positive charge at neutral pH such that the net charge of the therapeutic agent is greater than or equal to about +1;
the concentration of the plurality of particles during incubation is in the range of about 100 mg/mL to about 300 mg/mL;
the concentration of the therapeutic agent during incubation is in the range of about 10 mg/mL to about 20 mg/mL; and
the plurality of particles are incubated at a temperature in the range of about 35° C. to about 45° C.

43. (canceled)

44. (canceled)

45. (canceled)

46. (canceled)

47. (canceled)

48. (canceled)

49. (canceled)

50. (canceled)

51. (canceled)

52. (canceled)

53. (canceled)
